SEQUENCE LISTING (1 sequence)

SEQ ID NO: 1 - RNA, 22 nt, Homo sapiens
ugagaacugaauuccauggguu

(19) United States
(12) Patent Application Publication
(10) Pub. No.: US 2021/0338718 A1
(43) Pub. Date: Nov. 4, 2021

(54) METHODS FOR PREVENTING AND TREATING PULMONARY INFLAMMATION AND FIBROSIS

(71) Applicant: THE REGENTS OF THE UNIVERSITY OF COLORADO, A BODY CORPORATE, Denver, CO (US)

(72) Inventors: Kenneth LIECHTY, Denver, CO (US); Carlos ZGHEIB, Aurora, CO (US); Sarah Ashley HILTON, Denver, CO (US)

(21) Appl. No.: 17/281,049

(22) PCT Filed: Sep. 27, 2019
(86) PCT No.: PCT/US2019/053418

§ 371 Date: Mar. 29, 2021

Related U.S. Application Data
(60) Provisional application No. 62/738,352, filed on Sep. 28, 2018.

(51) Int. Cl. A61K 33/244; A61K 9/00; A61K 47/69; A61K 47/54; C12N 15/113; A61P 11/00

(52) U.S. Cl. CPC A61K 33/244; A61K 9/0043; A61K 47/6929; B82Y 5/00; A61K 47/6923; C12N 15/113; A61P 11/00; A61K 47/549

(57) ABSTRACT

Methods of treating, reducing the risk of, preventing, or alleviating a symptom of a pulmonary disease or condition, reducing or suppressing inflammation in the lung, and promoting lung repair, by pulmonary administration of a cerium oxide nanoparticle composition.

### TECHNICAL FIELD

This invention relates generally to uses of cerium oxide nanoparticles and/or active ingredients, in regulating, preventing and/or treating pulmonary injury, and pulmonary injury-related diseases.

A sequence listing submitted as an ASCII text file is hereby incorporated by reference.

### BACKGROUND

Lungs consist of bronchi, bronchioles, alveolar ducts and alveoli and function to exchange gas, including the transportation of oxygen from the air to the blood and the release of carbon dioxide from the blood to the outside of body. Reduction or loss of pulmonary function not only affects the entire respiratory system but also affects the body's water metabolism, blood circulation, and immune system. Reduction or loss of pulmonary function may become chronic obstructive pulmonary disease (COPD), idiopathic pulmonary fibrosis (IPF), emphysema, chronic bronchitis (CB), acute respiratory distress syndrome (ARDS), bronchopulmonary dysplasia (BPD), bronchiolitis obliterans (BO), and/or cryptogenic organizing pneumonia (COP).

Acute Lung Injury (ALI) and its more severe form, Acute Respiratory Distress Syndrome (ARDS), are characterized by an acute inflammatory response localized to the air spaces and lung parenchyma of the lungs. ALI and ARDS are major causes of acute respiratory failure and are associated with high morbidity and mortality in critically ill patients. ARDS may account for 36,000 deaths per year in a country the size of the US. Despite advances in ALI and ARDS patient management, such as lung-protective ventilation, there still exists a need for effective treatments.

Because current treatments are largely inefficient to improve survival of patients suffering from pulmonary disease, such as pulmonary inflammation and fibrosis, alternative therapies are urgently needed. The present invention addresses such needs.

### SUMMARY

This disclosure provides methods of treating, reducing the risk of, preventing, or alleviating a symptom of a pulmonary disease or condition in a subject, by administering to the subject a therapeutically effective amount of a cerium oxide nanoparticle (also referred to as “CeO₂ nanoparticles,” “nanoceria,” or “CNPs”) administered in a formulation for pulmonary administration. The CNP in the formulations of this disclosure may comprise a microRNA (miR or miRNA), which are small noncoding RNA molecules involved in the posttranscriptional regulation of gene expression. miR regulate the inflammatory response at multiple levels. In particular, miR-146a (SEQ ID NO. 1, having sequence ugagaacugaauuccauggguu) acts as the “molecular brake” on the inflammatory response.

Thus, the CNP in the formulations of this disclosure may comprise miR-146a attached to, conjugated with, or embedded within (i.e., non-covalently associated with) the CNP, such that the miR-146a-conjugated CNPs act as an active agent or therapeutic agent that is incorporated in the pharmaceutical formulations of this disclosure.

This disclosure also relates to use of a pharmaceutical formulation comprising the CNP in the manufacture of a medicament for promoting lung repair in a subject. This disclosure also relates to a pharmaceutical formulation comprising the CNP for promoting lung repair in a subject.

This disclosure also relates to a pharmaceutical formulation comprising the CNP for treating, reducing the risk of, preventing, or alleviating a symptom of a pulmonary disease or condition, or for reducing or suppressing inflammation in the lung, or for promoting lung repair in a subject.

This disclosure further relates to a kit comprising a pharmaceutical composition of this disclosure for use in a method of treating, reducing the risk of, preventing, or alleviating a symptom of a pulmonary disease or condition, or of reducing or suppressing inflammation in the lung, or of promoting lung repair in a subject.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description and no limitation as to the scope of the present disclosure is intended by either the inclusion or non-inclusion of elements, components, etc. in this Summary. Other features and advantages of the invention will be apparent from the following detailed description and claims.

### BRIEF DESCRIPTION OF FIGURES

FIG. 1 shows the effect of intratracheal instillation of PBS (control) on lung fibrosis and architecture (Trichrome staining for collagen) and inflammation (CD45+ cells by immunohistochemistry) at 7 days after treatment.

FIG. 2 shows the effect of intratracheal instillation of bleomycin on lung fibrosis and architecture (Trichrome stain) and inflammation (CD45+ immunohistochemistry) at 7 days after treatment.

FIG. 3 shows the effect of intratracheal instillation of both bleomycin and CNP-miR146a on lung fibrosis and architecture (Trichrome stain) and inflammation (CD45+ immunohistochemistry) at 7 days after treatment.

FIG. 4 shows the effect of intratracheal instillation of PBS, bleomycin, or both bleomycin and CNP-miR146a on lung fibrosis and architecture (Trichrome stain) and inflammation (CD45+ immunohistochemistry) at 14 days after treatment.

FIG. 5 shows the effect of intratracheal instillation of PBS or CNP-miR146a, 3 days after treatment of the lungs with bleomycin, on lung fibrosis and architecture (Trichrome stain) at 14 days after injury.

FIG. 6 shows the effect of intratracheal instillation of PBS or CNP-miR146a, 7 days after treatment of the lungs with bleomycin, on lung fibrosis and architecture (Trichrome stain) at 14 days after injury.

FIGS. 7A-7C comprise graphs showing the production of the pro-inflammatory cytokines Interleukin-6 (IL-6; FIG. 7A), Tumor Necrosis Factor (TNF; FIG. 7B), and Interleukin-1b (IL-1b; FIG. 7C) using qPCR at 7 days in lungs treated with PBS, bleomycin, or bleomycin with CNP-miR146a.

FIGS. 8A and 8B comprise graphs showing the expression of IL-6 and Irak1, respectively, only 3 days after bleomycin-induced injury.

FIG. 9 is a graph showing the impact of bleomycin and CNP-miR146a on the production of reactive oxygen species, indicated by the level of nitroxide measured in tissue samples after co-administration of bleomycin and CNP-miR146a and bleomycin application followed by CNP-miR146a treatment initiated at day 3.

FIGS. 10A and 10B comprise graphs showing interstitial macrophage and alveolar macrophage populations, respectively, present 10 days after bleomycin-induced injury with and without CNP-miR146a treatment.

FIG. 11 is a graph showing the impact of bleomycin and CNP-miR146a on macrophage recruitment at day 3 post-injury.

FIG. 12 is a graph showing lung injury severity scores determined via histological analysis of harvested tissue after bleomycin-induced injury with and without CNP-miR146a treatment.

FIG. 13 is a graph showing lung inspiratory capacity measured after treatment with bleomycin and/or CNP-miR146a.

FIG. 14 is a graph showing the effects of bleomycin and CNP-miR146a on tissue elastance.

FIG. 15 is a graph showing the effects of bleomycin and CNP-146a on tissue resistance.

FIGS. 16A and 16B comprise graphs of pulmonary volume (PV) loops showing the effects of bleomycin and CNP-miR146a on lung volume and pressure during inhalation and exhalation.

FIG. 17 is a graph showing miR146a expression measured over time in harvested lung tissue following bleomycin-induced injury.

### DETAILED DESCRIPTION

This disclosure relates to a method of treating, reducing the risk of, preventing, or alleviating a symptom of a pulmonary disease or condition, a method of reducing or suppressing inflammation in the lung, and a method of promoting lung repair by administering a cerium oxide nanoparticle formulation to the lung of a subject in need of such treatment.

Unless otherwise defined, all technical and scientific terms used herein have the same meaning as commonly understood by one of ordinary skill in the art to which this invention belongs. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In addition, the materials, methods, and examples are illustrative only and are not intended to be limiting.

A cerium oxide nanoparticle (also referred to as “CeO₂ nanoparticles,” “nanoceria,” or “CNP”) is an especially useful active agent in the pharmaceutical formulations of this disclosure. The production of such cerium oxide nanoparticles has been described in, for example, Chigurupati, et al., Biomaterials 34(9):2194-2201 (2013); and U.S. Pat. No. 7,534,453, which is incorporated herein by reference in its entirety. The CNPs in these pharmaceutical formulations may have a size range of about 2-10 nm, and in particular about 3-5 nm. These CNPs may be covalently conjugated to, or otherwise incorporate (i.e., non-covalently imbedded in or associated with), additional therapeutic agents (for example, micro RNA molecules, as described below). CNPs, including those containing additional active agents, are referred to herein as CNP compositions of this disclosure.

Another useful active agent in these formulations is microRNA (miR or miRNA), which are small noncoding RNA molecules involved in the post-transcriptional regulation of gene expression. miR regulate the inflammatory response at multiple levels. In particular, miR-146a acts as the “molecular brake” on the inflammatory response by targeting and repressing the activation of the NFκB inflammatory pathway. Thus, formulations of this disclosure may comprise miR-146a. These miR-146a active agents may be further conjugated to the CNPs described above, such that the miR-146a-conjugated CNPs (“CNP-146a”) act as an active agent or therapeutic agent in the formulations of this disclosure. The detailed synthesis and characterization of CNP conjugated to miRNA-146a has been described in PCT Publication No. WO 2017/091700, with international filing date of 23 Nov. 2016, which is incorporated by reference herein in its entirety. Briefly, oligonucleotides (i.e. miRNA-146a) contain phosphate groups carrying a negative charge along the chain that can electrostatically interact with the positively charged surface of the CNPs. In addition, oligonucleotides have hydroxyl groups of ribose and amino groups available for conjugation with the CNPs. The terminal functional group (amino, thiol, azide) for conjugation is also an option that may be utilized. Providing an appropriate excess of oligonucleotide in reaction medium (basically 10-15 molecules per nanoparticle), conjugation can be accomplished via different reactions. For example, amino groups of an oligonucleotide can be coupled with CNP hydroxyl groups or functional groups of CNP coating after their activation with carbodiimide (CDI), or other bifunctional activating agents. Unbound compounds, as well as by-products, can be removed by centrifugation at 8000 g for 10 min and by dialysis against water or PBS using mini dialysis columns with at least 20 kDa cut off.

As used herein, “subject” means a human or other mammal. Preferably, the subject is a human. A subject can be considered to be in need of treatment.

As used herein, a “pharmaceutically-acceptable excipient” or a “pharmaceutically-acceptable carrier” means a pharmaceutically acceptable material, composition, or vehicle involved in giving form or consistency to the pharmaceutical composition. Each excipient or carrier must be compatible with the other ingredients of the pharmaceutical composition when comingled such that interactions which would substantially reduce the efficacy of the active CNP compositions of this disclosure when administered to a subject and interactions which would result in pharmaceutical compositions that are not pharmaceutically acceptable are avoided. In addition, each excipient or carrier must of course be of sufficiently high purity to render it pharmaceutically-acceptable.

As used herein, “promoting” or “promote” means reducing the time for the lung to repair or recover from injuries or damages to the lungs or increasing the extent of lung repair or recovery. These formulations may promote lung repair or recovery by reducing or suppressing inflammation in the lungs.

As used herein, “suppressing”, “suppress”, or “suppression” means stopping the inflammation from occurring, worsening, persisting, lasting, or recurring.

“Reducing”, “reduce”, or “reduction” means decreasing the severity, frequency, or length of the inflammation.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a pulmonary disease or disorder if, after receiving a therapeutic amount of a CNP composition, according to the methods of this disclosure, the subject shows observable and/or measurable reduction in, or absence of, one or more of respiratory distress, oxygen requirement, ventilator dependence, and inflammatory markers. Alternatively or additionally, the subject may show an improvement in pulmonary function with reduced lung stiffness and improved compliance.

Reduction of these signs or symptoms may also be felt by the patient. The above parameters for assessing successful treatment and improvement in the pulmonary diseases and disorders are readily measurable by routine procedures familiar to a medical provider.

An “effective amount” of a CNP composition of this disclosure is an amount sufficient to carry out a specifically stated purpose. An “effective amount” may be determined empirically and in a routine manner, in relation to the stated purpose. The term “therapeutically effective amount” refers to an amount of a CNP composition, to “treat” a disease or disorder in a subject.

### Therapeutic Methods

The formulations containing CNP compositions described herein, e.g., CNP-146a, are suitable for the treatment of, reducing the risk of, prevention of, or alleviation of a symptom of a variety of pulmonary diseases or conditions. Pulmonary diseases and conditions are those that negatively affect the pulmonary or lung system in the body. Without intending to be bound by theory, CNP compositions of this disclosure are reactive oxygen species scavengers and are rapidly taken up by epithelial cells, decreasing the permeability of the lung, and/or suppressing the movement of leukocytes or fibrocytes from circulation to inflamed tissues. These CNP compositions may also improve cell viability and cell regeneration at the alveolar level.

The pharmaceutical formulations of this disclosure, e.g., CNP-146a, are suitable for treating, reducing the risk of, preventing, or alleviating a symptom of pulmonary diseases or conditions caused by or associated with inflammation, autoimmune diseases such as scleroderma and rheumatoid arthritis, Acute Lung Injury (ALI), Acute Respiratory Distress Syndrome (ARDS), blood clots in the lungs (pulmonary embolism), congestive heart failure, extended periods of low oxygen levels in the blood, and/or various medications and substances of abuse. In one embodiment, the pulmonary diseases or conditions are caused by or associated with inflammation of the lungs. In another embodiment, the pulmonary diseases or conditions are caused by or associated with ALI or ARDS.

In one embodiment, the pharmaceutical formulations of this disclosure, e.g., CNP-146a, are suitable for treating, reducing the risk of, preventing, or alleviating a symptom of pulmonary diseases or conditions caused by or associated with damages or injuries to the lungs. The damages or injuries to the lungs may be the result of use of medications, substance abuse, a medical condition, exposure to a pollutant or toxicant. The damage or injury to the lungs may cause inflammation to the lungs.

In one embodiment, the pharmaceutical formulations of this disclosure, e.g., CNP-146a, are suitable for treating, reducing the risk of, preventing, or alleviating a symptom of pulmonary diseases or conditions caused by or associated with narrowing of pulmonary blood vessels. The narrowing of pulmonary blood vessels may be a result of the use of medications, substance abuse, or a medical condition. The narrowing of pulmonary blood vessels (e.g., arteries, veins, and capillaries) may cause a decrease in the amount of blood that flows through the blood vessels, and/or an increase in the pressure of the blood that flows through pulmonary blood vessels.

Pulmonary diseases and conditions that may be treated using the pharmaceutical formulations and methods of this disclosure include, but are not limited to, chronic obstructive pulmonary disease (COPD), emphysema, asthma, idiopathic pulmonary fibrosis, pneumonia, tuberculosis, cystic fibrosis, bronchitis, pulmonary hypertension (e.g., Idiopathic Pulmonary Arterial Hypertension (IPAH) (also known as Primary Pulmonary Hypertension (PPH)) and Secondary Pulmonary Hypertension (SPH)), interstitial lung disease, and lung cancer.

An interstitial lung disease occurs when the interstitial tissue, which lines alveoli in the lungs, becomes scarred. Scarring causes inflammation of these tissues, affecting their ability to absorb oxygen. Causes of interstitial lung disease include, but are not limited to, environmental pollutants, lung tissue injury resulting from trauma or infection, and various connective tissue diseases.

Asthma affects millions of individuals around the world, from children to senior citizens. Asthma is caused by the contraction of the muscles in the airway, excessive mucus production, and swelling or inflammation of the airways or branches of the lungs. Airway constriction and inflammation results in reduced air flow to the lungs, which can often be noted by the wheezing sounds a person having an asthma attack may make. The treatment and management of asthma is determined on an individualized basis and is subject to considerations including the severity and frequency of asthma attacks experienced by the patient.

Bronchitis is a chronic infection of the bronchioles in the lungs. The bronchioles contain the alveoli, which are responsible for gas exchange during respiration. When bronchioles become infected, the immune system response results in swelling and increased mucous production in the airways, making it difficult to breathe. Bronchitis is also presented with a chronic, painful cough.

Emphysema also affects the alveoli, to the extent at which the cells that make them up are completely destroyed. Emphysema also destroys villi in the lungs. Villi are hair-like structures that push foreign substances out of the lungs. When villi are destroyed, the lungs have an increased chance of infection. The effects of emphysema are permanent and result in life long breathing difficulties.

One of the most common forms of COPD is emphysema. COPD damages the alveoli in the lungs, which are small air sacs found at the end of the lung branches that transport oxygen to the sacs. Weakened sac walls inhibit adequate oxygen flow into and out of the sacs, causing constant shortness of breath.

Cystic fibrosis is another common pulmonary disease that is hereditary in nature, meaning the condition is often passed down through family lines. A gene mutation causes the lungs to absorb excessive amounts of water and sodium, resulting in a buildup of fluids in the lungs that decreases their ability to absorb enough oxygen for optimal function. This condition gradually worsens as lung cells become increasingly damaged and eventually die.

Idiopathic pulmonary fibrosis (IPF) (or cryptogenic fibrosing alveolitis (CFA)) is a chronic, progressive form of lung disease characterized by fibrosis of the supporting framework (interstitium) of the lungs. By definition, the term is used only when the cause of the pulmonary fibrosis is unknown (“idiopathic”).

Tuberculosis is a disease that can spread from person to person through the air. It is a bacterial infection of the lungs. Anti-tuberculosis drugs are needed to kill bacteria very effectively. However, some strains of tuberculosis have developed a resistance to the anti-bacterial drugs used for treatment of the disease.

The CNP compositions described herein, e.g., CNP-146a, are suitable for the treatment, reducing the risk of, prevention, or alleviation of a symptom of an interstitial lung disease, asthma, bronchitis, COPD, emphysema, cystic fibrosis, IPF, tuberculosis, or pulmonary hypertension (e.g., IPAH, PPH, and SPH).

The pulmonary formulations of this disclosure, e.g., CNP-146a, are also suitable for reducing or suppressing inflammation in the lungs. Without intending to be bound by theory, pulmonary formulations of this disclosure reduce or suppress inflammation by decreasing the permeability of the lung and/or suppressing the movement of leukocytes or fibrocytes from circulation to inflamed tissues. The reduction and/or suppression of inflammation is evidenced by a decrease in the number CD45+ cells observed in injured lung tissue treated with the disclosed pulmonary formulations, e.g., CNP-146a, before, concurrently with, or after, the lung injury.

The pulmonary formulations of this disclosure, e.g., CNP-146a, are also suitable for promoting lung repair or recovery. In one embodiment, the CNP compositions of this disclosure decrease the number of fibrocytes moved to the lungs or to the location of injury in the lungs from circulation. This may include decreasing the amount of a protein, a peptide, or a chemokine produced by the fibrocytes in the lungs or at the location of injury in the lungs. The pulmonary formulations disclosed herein, e.g., CNP-146a, may decrease the number of interstitial macrophages present in injured lung tissue, while also increasing the number of alveolar macrophages. Total macrophage numbers recruited to the site(s) of lung injury may also decrease after treatment with such pulmonary formulations.

In one embodiment, the CNP compositions of this disclosure, e.g., CNP-146a, regulate the expression of a gene involved in inflammation, for example by decreasing the expression of a pro-inflammatory factor, such as decreasing the expression of IL-6, TNF, Irak1, and/or IL-1b. In a related embodiment, the CNP compositions of this disclosure, e.g., CNP-146a, inhibit the infiltration and accumulation of CD45+ cells, as mentioned above. Embodiments of the CNP compositions of this disclosure, e.g., CNP-146a, may also drive a reduction in the presence of reactive oxygen species in injured lung tissue.

By treating lung tissue with the pulmonary formulations described herein, e.g., CNP-146a, damaged lung tissue may exhibit increases in inspiratory capacity, along with decreases in tissue elastance and resistance, thereby also improving compliance.

The formulations of this disclosure may be administered to a subject before or after a lung injury. Typically, the formulations of this disclosure are administered to a subject after a lung injury.

### Pharmaceutical Formulations

CNP compositions of this disclosure, e.g., CNP-146a, may be administered as a pharmaceutical formulation. A CNP compound of this disclosure and a pharmaceutically-acceptable excipient or excipients will typically be formulated into a dosage form adapted for pulmonary or nasal administration to the subject. For example, dosage forms may include those adapted for inhalation such as aerosols, solutions, and dry powders.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform aerosol for inhalation. Alternatively or additionally, certain pharmaceutically-acceptable excipients may be chosen for their ability to facilitate the production of stable dosage forms. Alternatively or additionally, certain pharmaceutically-acceptable excipients may be chosen for their ability to enhance compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in this disclosure. In addition, there are resources available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of this disclosure are prepared using techniques and methods known to those skilled in the art. Some of the methods commonly used in the art are described in Remington's Pharmaceutical Sciences (Mack Publishing Company).

The CNP compositions of this disclosure may also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxidepolylysine substituted with palmitoyl residues. Furthermore, the CNP compositions of this disclosure may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Thus, one aspect of this disclosure is oral inhalation or intranasal administration of CNP-containing compositions. Appropriate dosage forms for such administration, such as an aerosol formulation or a metered dose inhaler, may be prepared by conventional techniques.

For administration by inhalation, the CNP compositions may be delivered in the form of an aerosol spray from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of a CNP compound of this disclosure and a suitable powder base such as lactose or starch.

Dry powder compositions for topical delivery to the lung by inhalation may, for example, be presented in capsules and cartridges of for example gelatin, or blisters of for example laminated aluminum foil, for use in an inhaler or insufflator. Powder blend formulations generally contain a powder mix for inhalation of the CNP compound of this disclosure and a suitable powder base (carrier/diluent/excipient substance) such as mono-, di- or poly-saccharides (e.g., lactose or starch). Each capsule or cartridge may generally contain between 20 μg-10 mg of the CNP compositions of this disclosure, optionally in combination with another therapeutically active ingredient. Alternatively, the CNP compound of this disclosure may be presented without excipients.

Suitably, the packing/medicament dispenser is of a type selected from the group consisting of a reservoir dry powder inhaler (RDPI), a multi-dose dry powder inhaler (MDPI), and a metered dose inhaler (MDI).

A reservoir dry powder inhaler (RDPI) is an inhaler having a reservoir form pack suitable for comprising multiple (un-metered) doses of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the subject for inhalation.

A multi-dose dry powder inhaler (MDPI) is an inhaler suitable for dispensing medicament in dry powder form, wherein the medicament is located within a multi-dose pack containing (or otherwise carrying) multiple, defined doses (or parts thereof) of the CNP composition medicament. The carrier may be a blister pack form, but it may also be a capsule-based pack form or a carrier onto which medicament has been applied by any suitable process including printing, painting, and vacuum occlusion.

In the case of multi-dose delivery, the formulation can be pre-metered (e.g., as in Diskus, see GB 2242134, U.S. Pat. Nos. 6,632,666, 5,860,419, 5,873,360 and 5,590,645, or Diskhaler, see GB 2178965, 2129691 and 2169265, U.S. Pat. Nos. 4,778,054, 4,811,731, and 5,035,237, the disclosures of each of which are hereby incorporated by reference) or metered in use (e.g., as in Turbuhaler, see EP 69715 or in the devices described in U.S. Pat. No. 6,321,747, the disclosures of each of which are hereby incorporated by reference). An example of a unit-dose device is Rotahaler (see GB 2064336 and U.S. Pat. No. 4,353,656, the disclosures of each of which are hereby incorporated by reference).

The Diskus inhalation device comprises an elongate strip formed from a base sheet having a plurality of recesses spaced along its length and a lid sheet hermetically sealed thereto to define a plurality of containers, each container having therein an inhalable formulation containing a CNP compound of this disclosure, optionally combined with lactose. The strip is sufficiently flexible to be wound into a roll. The lid sheet and base sheet will preferably have leading end portions which are not sealed to one another and at least one of the said leading end portions is constructed to be attached to a winding means. Also, the hermetic seal between the base and lid sheets extends over their whole width. The lid sheet may preferably be peeled from the base sheet in a longitudinal direction from a first end of the said base sheet.

The multi-dose pack may be a blister pack comprising multiple blisters for containment of medicament in dry powder form. The blisters are typically arranged in regular fashion for ease of release of medicament therefrom.

The multi-dose blister pack may comprise a plurality of blisters arranged in generally circular fashion on a disc-form blister pack. In another embodiment, the multi-dose blister pack is elongate in form, for example, comprising a strip or a tape.

The multi-dose blister pack may be defined between two members secured to one another. U.S. Pat. Nos. 5,860,419, 5,873,360 and 5,590,645 describe medicament packs of this general type. The device is usually provided with an opening station comprising peeling means for peeling the members apart to access each medicament dose. Suitably, the device is adapted for use where the members are elongate sheets which define a plurality of medicament containers spaced along the length thereof, the device being provided with indexing means for indexing each container in turn. Also, the device may be adapted for use wherein one of the sheets is a base sheet having a plurality of pockets therein, and the other of the sheets is a lid sheet, each pocket and the adjacent part of the lid sheet defining a respective container, the device having driving means to pull the lid sheet and base sheet apart at the opening station.

A metered dose inhaler (MDI) is a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the subject. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable. The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 75 μl. In one aspect, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a “free flow aerosol valve” having a chamber and a valve stem extending into the chamber and movable relative to the chamber between dispensing and non-dispensing positions. The valve stem has a configuration and the chamber has an internal configuration such that a metered volume is defined there between and during movement between the non-dispensing and dispensing positions, the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

Additionally, intra-nasal delivery of the CNP compositions of this disclosure is also effective. To formulate an effective pharmaceutical nasal composition, the medicament must be delivered readily to all portions of the nasal cavities (the target tissues) where it performs its pharmacological function. Additionally, the CNP compositions should remain in contact with the target tissues for relatively long periods of time. The CNP compositions may be capable of resisting those forces in the nasal passages that function to remove particles from the nose. Such forces, referred to as “mucociliary clearance”, are recognized as being extremely effective in removing particles from the nose in a rapid manner, for example, within 10-30 minutes from the time the particles enter the nose.

Nasal compositions preferably i) do not contain ingredients which cause the user discomfort, ii) have satisfactory stability and shelf-life properties, and iii) do not include constituents that are considered to be detrimental to the environment, for example compounds that deplete ozone. Typically, when administered to the nose, the subject inhales deeply subsequent to the nasal cavity being cleared. During inhalation, the formulation would be applied to one nostril while the other is manually compressed. This procedure is then repeated for the other nostril.

Another means for applying the formulation of the present invention to the nasal passages is by use of a pre-compression pump (such as the pre-compression pump VP7 model manufactured by Valois SA). Such pumps are beneficial to ensure that the formulation is not released until a sufficient force has been applied, otherwise smaller doses may be applied. Another advantage of the pre-compression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-100 μl of the formulation.

Spray compositions for topical delivery to the lung by inhalation may be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the CNP compositions of this disclosure optionally in combination with another therapeutically active ingredient and a suitable propellant such as a fluorocarbon or hydrogen-containing chlorofluorocarbon or mixtures thereof, particularly hydrofluoroalkanes, e.g. of any of which may include dichlorodifluoromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g., an aluminum canister) closed with a valve (e.g., a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes, the particles of the CNP active ingredient as produced may be reduced in size by conventional means, e.g., by micronization.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product.

The following Examples are illustrative and should not be interpreted in any way so as to limit the scope of the invention.

### EXAMPLES

Bronchopulmonary dysplasia and chronic lung disease due to prematurity are major causes of morbidity and mortality in pre-term infants. Central to the pathogenesis of bronchopulmonary dysplasia is chronic inflammation leading to fibrosis. In addition to inflammation there is significant oxidative stress. The inventors investigated whether a CNP therapeutic that targets inflammation and oxidative stress could reduce fibrosis and the long-term sequela these infants face. MicroRNA-146a was conjugated to cerium oxide nanoparticles (CNP-146a) because they are reactive oxygen species scavengers and rapidly taken up by epithelial cells. The inventors initiated intratracheal delivery of CNP-146a both at the time of bleomycin injury and at defined periods thereafter to evaluate its effects on preventing and/or decreasing pulmonary inflammation and subsequent fibrosis.

To evaluate the effects of CNP-146a on lung injuries induced by bleomycin, which is a pulmonary toxin, 29 young (10 week) male and female C57BL/6 mice were anesthetized and treated with intratracheal instillation of bleomycin (3 units/kg), PBS (as a control) or bleomycin and a single dose of CNP-146a (10 uM) at various timepoints. Half of the animals were euthanized at 7 days for bronchial alveolar lavage and tissue harvest, and half were euthanized at 14 days for lung inflation and tissue harvest. Histological analysis of the harvested samples was performed by staining the connective tissues, e.g., collagen, with Trichrome. The production of CD45, an inflammation marker, was measured via immunohistochemistry, and the expression of multiple inflammatory cytokines was measured via quantitative PCR (qPCR). Increased expression of such markers is a typical indication of acute lunge injury (ALI). Various parameters of lung tissue and lung function, e.g., tissue elastance and inspiratory capacity, were also measured.

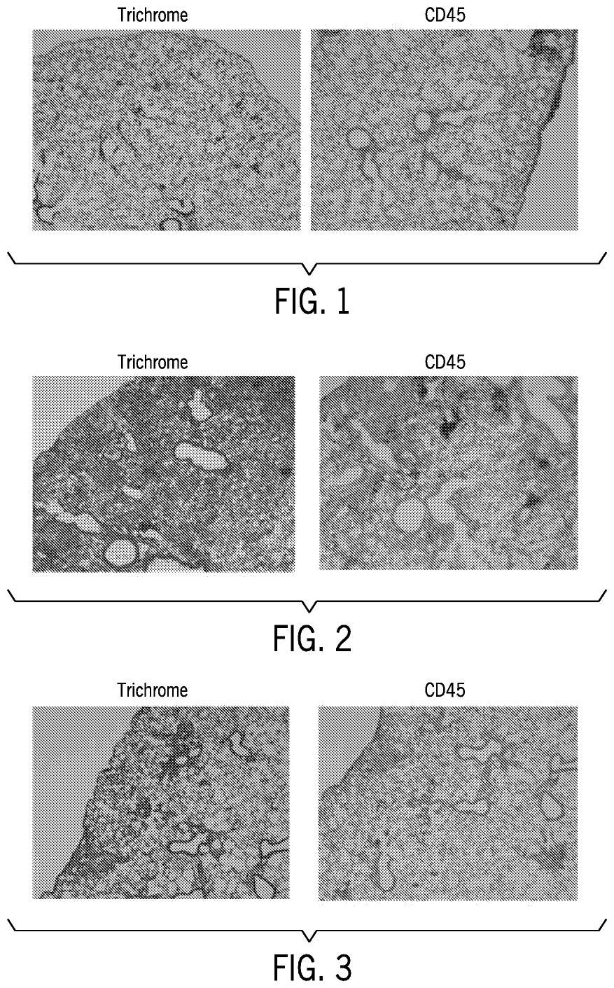

FIG. 1 shows the effects of intratracheal instillation of PBS (control) on lung fibrosis and architecture (Trichrome staining for collagen) and inflammation (CD45+ cells by immunohistochemistry) at 7 days after treatment. The connective tissue appears normal, as does CD45 expression. By contrast, FIG. 2 shows the effect of intratracheal instillation of bleomycin on lung fibrosis and architecture (Trichrome stain) and inflammation (CD45+ immunohistochemistry) at 7 days after treatment. Relative to FIG. 1, the tissue shown in FIG. 2 appears inflamed (left) and includes a greater number of CD45+ cells.

FIG. 3 shows the effects of intratracheal instillation of both bleomycin and CNP-146a on lung fibrosis and architecture (Trichrome stain) and inflammation (CD45+ immunohistochemistry) at 7 days after treatment. As shown, the study group treated with CNP-146a at the time of bleomycin injury exhibited less mucosal sloughing/hemorrhage, decreased inflammation evidenced by a reduction in CD45+ cells, and a marked decrease in lung fibrosis relative to the tissue treated with bleomycin, only. Bronchial alveolar lavage (BAL) specimens were also processed for cell viability. No cell viability was observed in the bleomycin treated group at 7 days (0.0% cell viability compared to 32.04% cell viability in the PBS controls). The CNP-146a treated group had improved cell viability equivalent to the control animals (35.92% cell viability).

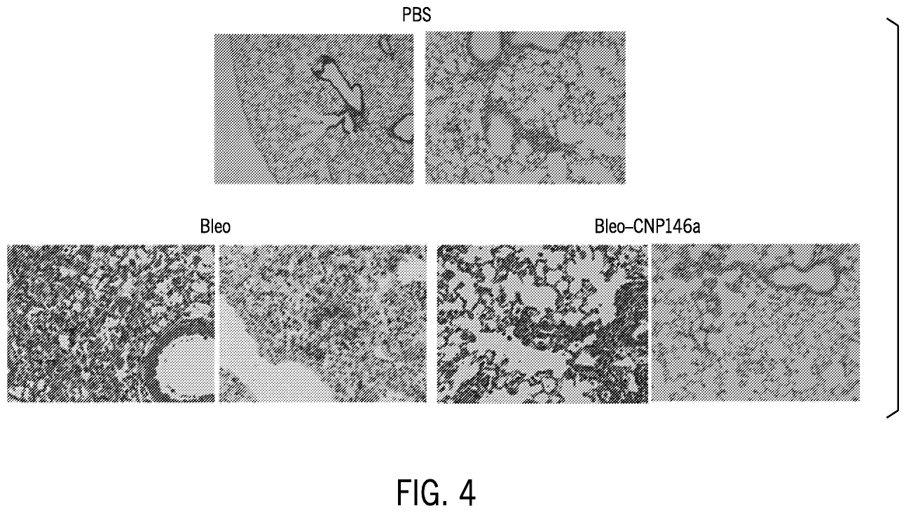

FIG. 4 shows the effects of intratracheal instillation of PBS, bleomycin, and both bleomycin and CNP-146a on lung fibrosis and architecture (Trichrome stain) and inflammation (CD45+ immunohistochemistry) at 14 days after treatment. Histological analysis at 14 days revealed significant lung injury caused by bleomycin, including alveolar hemorrhage, increased collagen deposition, and abundance of CD45+ cells compared to PBS controls. The study group treated with CNP-146a at the time of bleomycin injury showed less mucosal sloughing/hemorrhage, decreased inflammation evidenced by decreased CD45+ cells, and a marked decrease in lung fibrosis. Together, these observations indicate that CNP-146a may be effective to prevent or at least reduce the effects of lung injury.

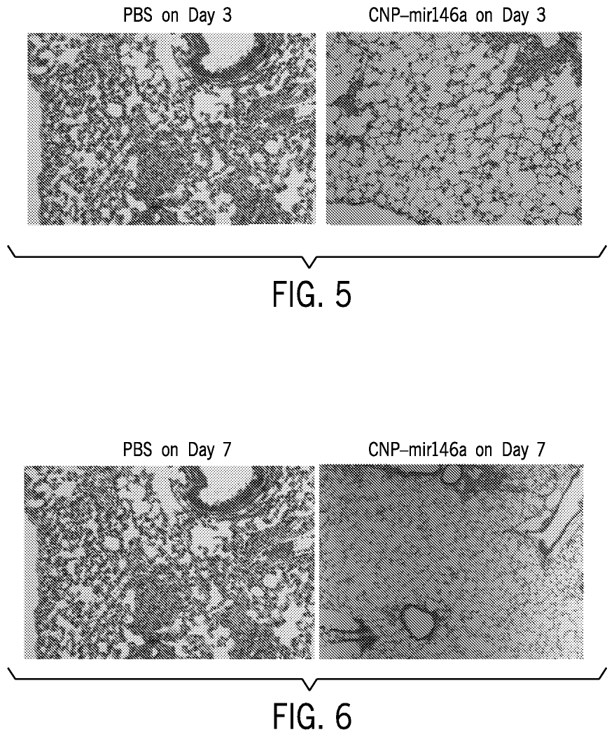

FIG. 5 shows the effects of intratracheal instillation of PBS or CNP-146a, administered 3 days after exposure of the lungs to bleomycin, on lung fibrosis and architecture (Trichrome stain) measured at 14 days after injury. As shown, treating the lungs with CNP-146a 3 days after injury reduced mucosal sloughing/hemorrhage and fibrosis caused by bleomycin, indicating that CNP-146a may be effective to reverse at least a portion of the effects caused by lung injury.

FIG. 6 shows the effects of intratracheal instillation of PBS or CNP-146a, administered 7 days after exposure of the lungs to bleomycin, on lung fibrosis and architecture (Trichrome stain) measured at 14 days after injury. Similar to the effects shown in FIG. 5, treating the lungs with CNP-146a 7 days after injury reduced mucosal sloughing/hemorrhage and fibrosis caused by bleomycin, further confirming that CNP-146a may be effective to reverse at least a portion of the effects associated with lung injury, even when administered a full week after the injury occurred.

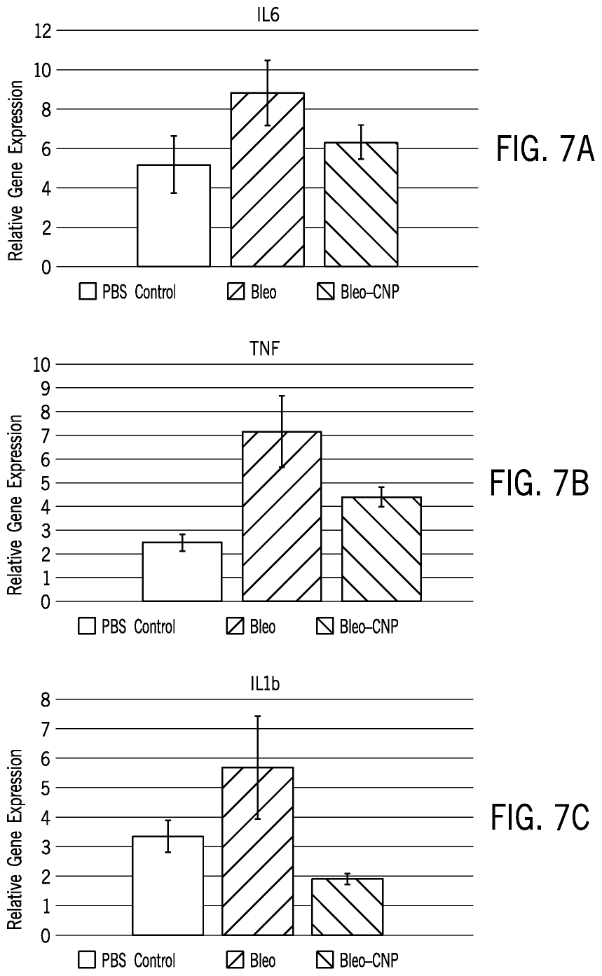

FIGS. 7A-7C show the production of the pro-inflammatory cytokines Interleukin-6 (IL-6; FIG. 7A), Tumor Necrosis Factor (TNF; FIG. 7B), and Interleukin-1b (IL-1b; FIG. 7C) using qPCR at 7 days in lungs treated with PBS, bleomycin, or bleomycin with CNP-146a. As shown, bleomycin resulted in a significant increase in the gene expression of IL-6, TNF, and IL-1b compared to PBS-treated lungs. The addition of CNP-146a to the bleomycin-exposed tissue resulted in a significant downregulation in the expression of these pro-inflammatory cytokines, similar to levels seen in the PBS-treated lungs, indicating that CNP-146a may effectively reduce the expression of cytokines implicated in driving inflammation.

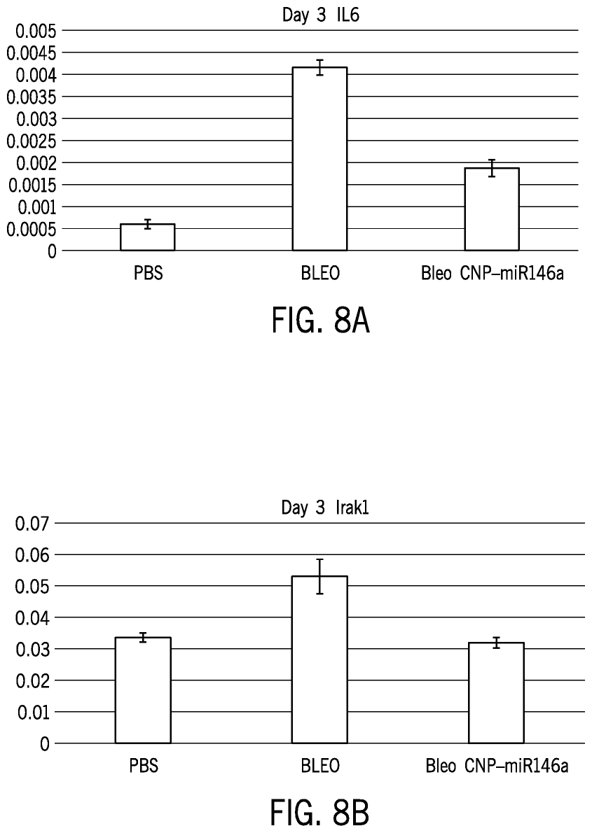

FIGS. 8A and 8B show the expression of IL-6 and Irak1, respectively, only 3 days after bleomycin-induced injury. Like IL-6, increased Irak1 expression is a common indication of inflammation. In response to bleomycin injury, expression of both IL-6 and Irak1 increased significantly by day 3 relative to the PBS control. Treatment with CNP-146a concurrently with bleomycin, however, maintained a lower level of IL-6 and Irak1 expression, further indicating that CNP-146a may be effective in preventing the effects associated with lung injury. Expression of Irak1, in particular, remained nearly identical to the expression measured in the PBS control samples.

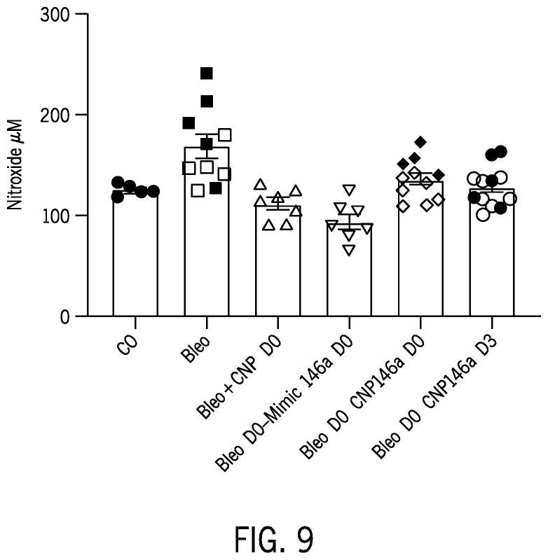

FIG. 9 shows the impact of bleomycin and CNP-146a on the production of reactive oxygen species, indicated by the level of nitroxide measured in the tissue samples after bleomycin-CNP146a co-administration, and bleomycin application followed by CNP-146a treatment at day 3. As shown, bleomycin increased nitroxide production relative to the control (CO). This effect was reduced by concurrent application of both bleomycin and CNP-146a at day 0. Bleomycin application at day 0 followed by CNP-146a treatment at day 3 also caused a reduction in nitroxide production relative to the tissue treated with bleomycin, only. This data shows that CNP-146a may be effective to not only prevent the effects of lung injury, but also reduce effects that have already occurred.

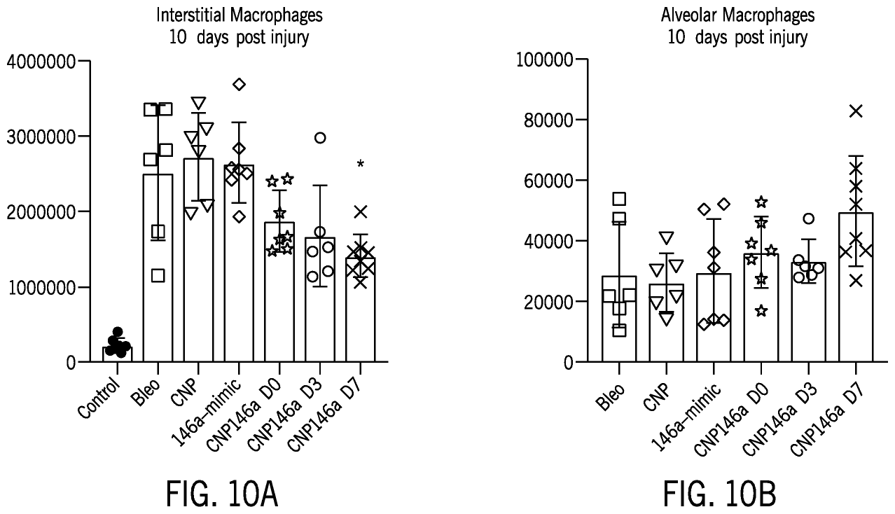

FIGS. 10A and 10B show the number of interstitial macrophages and alveolar macrophages, respectively, present in lung tissue 10 days after bleomycin-induced injury. Increased numbers of interstitial macrophages are often observed in areas of pulmonary inflammation, while alveolar macrophage populations typically decrease in inflamed tissue. As shown in FIG. 10A, bleomycin caused a marked increase in the number of interstitial macrophages, an effect that is reduced by CNP-146a treatment initiated at day 0, day 3, and especially day 7, indicating yet again that CNP-146a may prevent the onset of lung conditions induced by injury and reverse its effects. Consistent with this finding, FIG. 10B shows that the number of alveolar macrophages increased with CNP-146a treatment initiated concurrently with bleomycin at day 0, and CNP146a treatment initiated at day 3 and day 7 after bleomycin exposure.

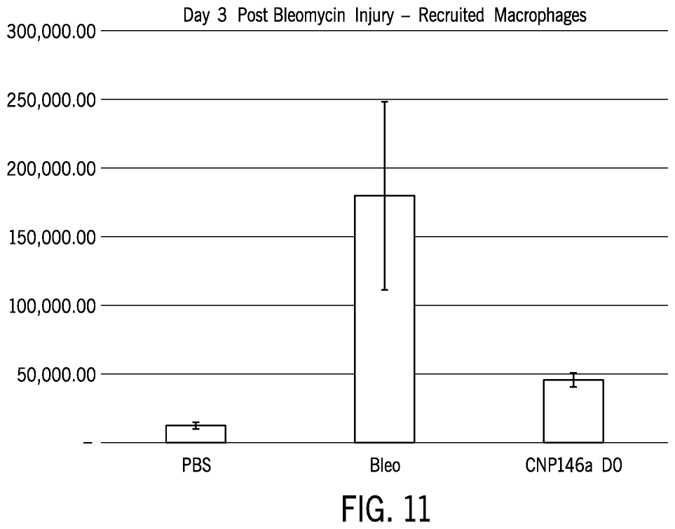

FIG. 11 shows the impact of bleomycin and CNP-146a on macrophage recruitment at day 3 post-injury. The number of macrophages recruited to a site of inflammation, infection, or injury typically increases. Bleomycin-induced injury significantly increased the level of macrophage recruitment, while concurrent CNP-146a treatment nearly eliminated the increase in macrophage recruitment, reaffirming the role of CNP-146a in preventing inflammation.

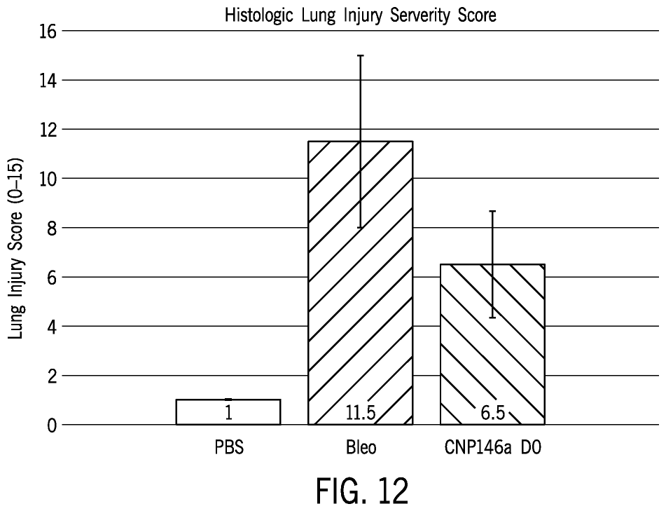

FIG. 12 is a representation of the overall lung injury severity score determined via histological analysis of harvested tissue after bleomycin-induced injury. As shown, the lung injury score determined after PBS treatment was 1, while the score increased to 11.5 after bleomycin-induced injury. Treatment with CNP-146a concurrently with bleomycin exposure at day 0 led to a score of only 6.5. Accordingly, CNP-146a treatment prevented the significant increase in lung injury caused by bleomycin.

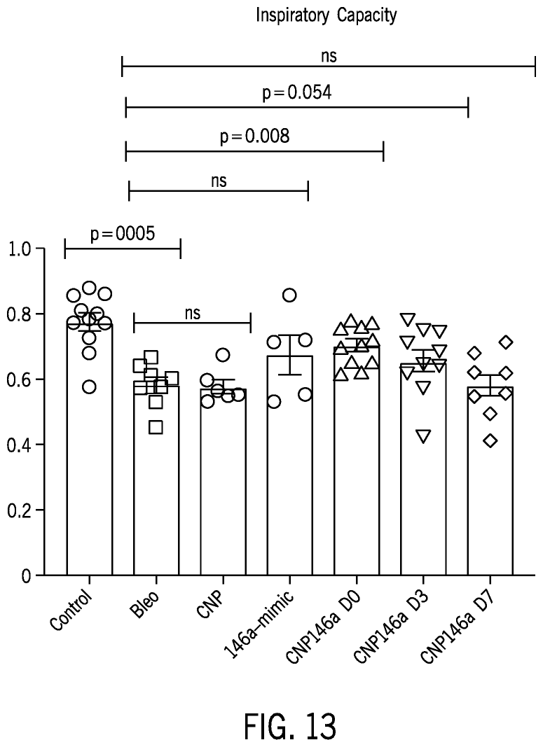

To evaluate the impact of CNP-146a on the more symptomatic effects of lung injury on tissue properties and functions, measurements of inspiratory capacity, tissue elastance, tissue resistance, and pulmonary volume were obtained. FIG. 13 is a graph showing inspiratory capacity after treatment with bleomycin and/or CNP-146a. Reduced inspiratory capacity is another indication of lung injury. Relative to the negative control, bleomycin decreased inspiratory capacity, an effect that was substantially reversed via concurrent CNP-146a treatment at day 0. The effect was also reduced by treatment with CNP-146a initiated at day 3 and day 7 post-injury.

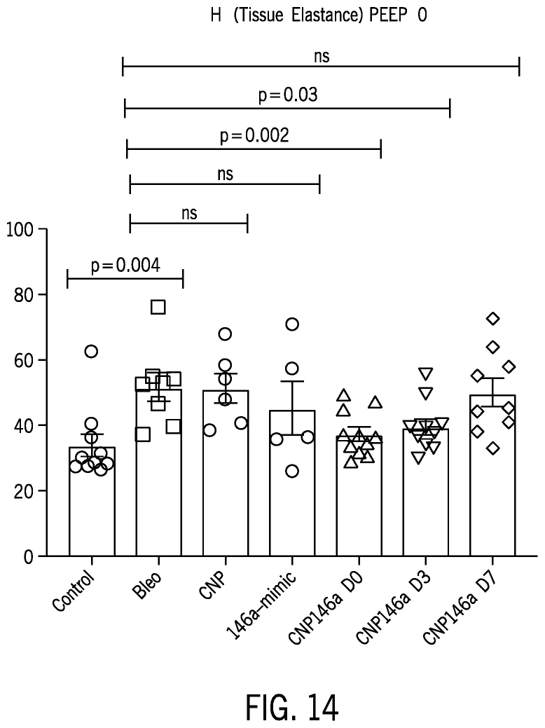

FIG. 14 shows the effects of bleomycin and CNP-146a on tissue elastance, which typically increases after injury. Indeed, bleomycin exposure increased lung tissue elastance relative to the negative control. Treatment with CNP-146a at day 0 reduced this effect, as did CNP-146a treatment initiated at days 3 and 7. Accordingly, CNP-146a treatment may prevent and reduce increases in elastic stiffness of lung tissue typically caused by damage.

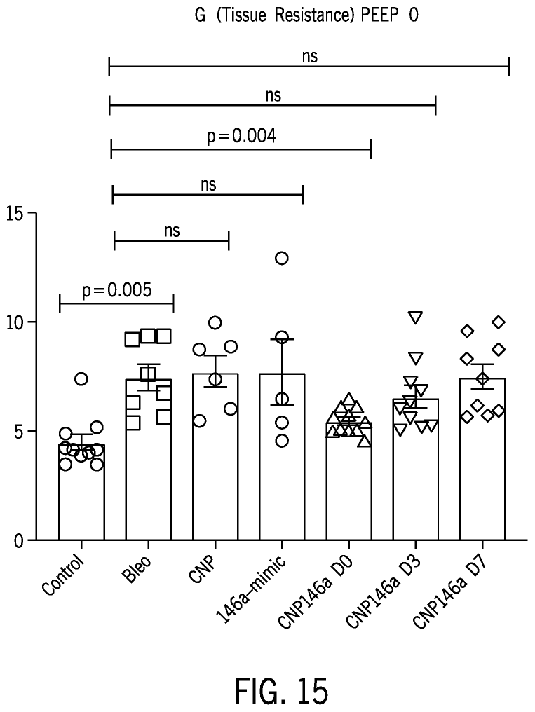

FIG. 15 shows the effects of bleomycin and CNP-146a on tissue resistance, another property that usually increases after injury. As shown, bleomycin application caused an increase in resistance. Treatment with CNP-146a reduced this effect, evidenced by decreased tissue resistance relative to the tissue exposed to bleomycin, only. CNP-146a treatment caused this effect when initiated at day 0, day 3, and day 7.

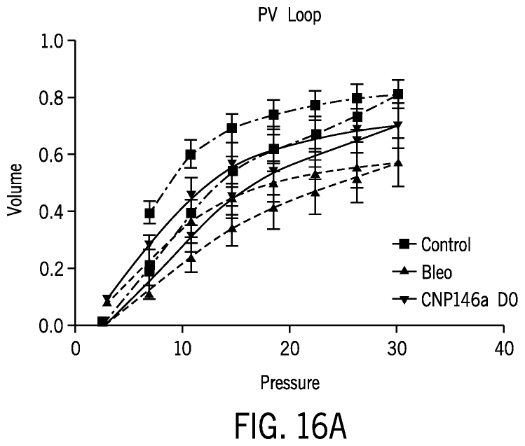

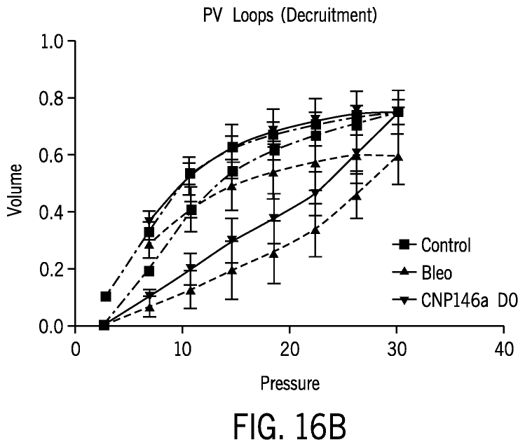

FIGS. 16A and 16B are graphs of pulmonary volume (PV) that show the effects of bleomycin and CNP-146a on lung volume and pressure during inhalation and exhalation. As shown by the PV loops of FIG. 16A, bleomycin exposure caused a consistent decrease in lung volume measured at the same time and pressure points as tissue not exposed to bleomycin. This effect was not as severe in tissues treated concurrently with CNP-146a, indicating that lung tissue stiffness may decrease, and maximum lung volume may increase, in response to CNP-146a treatment initiated concurrently with bleomycin injury. Similar effects are shown in FIG. 16B, which illustrates the effects of bleomycin and CNP-146a on lung pressure and volume during derecruitment.

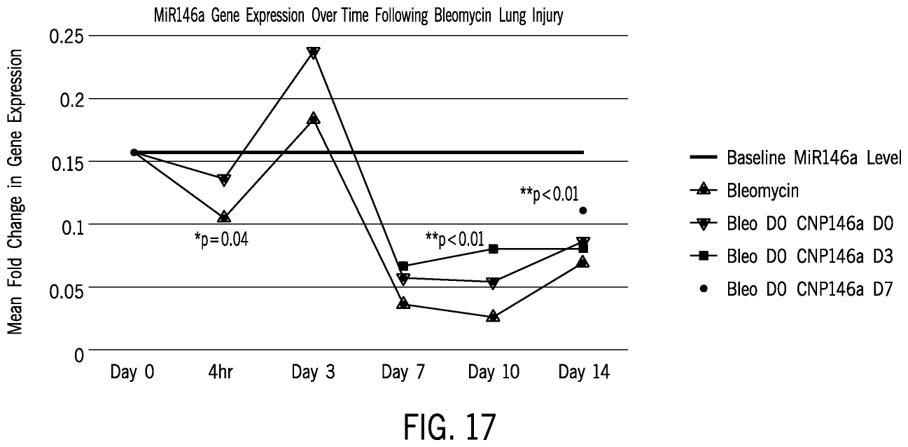

FIG. 17 shows miR146a expression measured over time in harvested lung tissue following bleomycin-induced injury and CNP-146a treatment. The graph shows a spike in miR146a expression peaking 3 days after CNP-146a administration, which drops until day 7 before substantially leveling off. The level of miR146a expression initiated at day 3 remained substantially consistent through day 14. These results suggest that CNP-146a administration may be most effective when administered as early as possible in the event of lung injury or infection.

The data described above demonstrate that CNP-146a is a new therapeutic that can be administered for the prevention and treatment of chronic pulmonary disease in children. The histology at 3, 7 and 14 days shows improvement in the bleomycin and CNP-146a treatment group. Because the treated lungs are not completely normal when compared to control, repeated dosing may be needed for complete treatment. Additionally, the BAL samples show only dead cells in the bleomycin group with improved cell viability in the CNP-146a group, indicating cell regeneration at the alveolar level. Overall, the decrease in inflammation and fibrosis seen with CNP-146a treatment suggests effective treatment of chronic pulmonary disease. While the effects of CNP-146a administration are described above in connection with a bleomycin-induced model of lung injury, similar effects can be shown in different lung injury models, including models of lipopolysaccharide-induced injury.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, or step is necessary or indispensable. The novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method of treating, reducing the risk of, preventing, or alleviating a pulmonary disease or condition in a subject, comprising administering to the subject a therapeutically effective amount of a cerium oxide nanoparticle (CNP).

2. The method of claim 1, wherein the CNP comprises a therapeutic agent.

3. The method of claim 1 or 2, wherein the therapeutic agent is an anti-inflammatory agent.

4. The method of claim 1 or 2, wherein the therapeutic agent is a micro-RNA (miRNA).

5. The method of any one of claims 1-4, wherein the miRNA is miRNA-146a.

6. The method of claim 4 or 5, wherein the miRNA is covalently attached to the CNP.

7. The method of claim 4 or 5, wherein the miRNA is non-covalently associated with the CNP.

8. The method of any one of claims 1-7, wherein the pulmonary disease or condition is selected from pulmonary inflammation, pulmonary fibrosis, obstructive pulmonary disease (COPD), emphysema, asthma, idiopathic pulmonary fibrosis, pneumonia, tuberculosis, cystic fibrosis, bronchitis, pulmonary hypertension (e.g., Idiopathic Pulmonary Arterial Hypertension (IPAH), interstitial lung disease, and lung cancer.

9. The method of claim 8, wherein the pulmonary disease or condition is selected from pulmonary inflammation, pulmonary fibrosis, COPD, emphysema, asthma, pulmonary fibrosis, and cystic fibrosis.

10. The method of any one of claims 1-7, wherein the pulmonary disease or condition is caused by inflammation, autoimmune disease, scleroderma, rheumatoid arthritis, Acute Lung Injury (ALI), Acute Respiratory Distress Syndrome (ARDS), pulmonary embolism, congestive heart failure, heart valve disease, extended periods of low oxygen levels in the blood, medication, or substance of abuse.

11. The method of claim 10, wherein the pulmonary disease or condition is caused by inflammation.

12. A method of reducing or suppressing inflammation in the lung in a subject, comprising administering to the lungs of a subject in need thereof a therapeutically effective amount of a CNP linked to miRNA-146a.

13. A method of promoting lung repair in a subject, comprising administering to the lungs of a subject in need thereof a therapeutically effective amount of a CNP linked to miRNA-146a.

14. The method of any one of claims 1-13, wherein the subject is a human.

15. The method of any one of claims 1-14, wherein the CNP is administered by inhalation.

16. The method of any one of claims 1-15, wherein the CNP is administered as an aerosol formulation.

17. The method of any one of claims 1-15, wherein the CNP is administered as a metered dose inhaler.

18. The method of any one of claims 1-15, wherein the CNP is administered from a nebulizer.

19. The method of any one of claims 1-15, wherein the CNP is administered as a dry powder composition for topical delivery to the lung by inhalation.

20. The method of any one of claims 1-15, wherein the CNP is administered from a reservoir dry powder inhaler (RDPI), a multi-dose dry powder inhaler (MDPI), or a metered dose inhaler (MDI).

21. The method of any one of claims 1-15, wherein the CNP is administered to nasal passages of the subject from a pre-compression pump.

22. The method of any one of claims 1-15, wherein the CNP is administered as an intranasal spray.
